(19) United States
(12) Patent Application Publication
(10) Pub. No.: US 2009/0306159 A1
(43) Pub. Date: Dec. 10, 2009

(54) Inhibition of Intermediate-Conductance Calcium Activated Potassium Channels in the Treatment and/or Prevention of Atherosclerosis

(75) Inventors: Heike Wulff, Davis, CA (US); George K. Chandy, Laguna Beach, CA (US); Hiroto Miura, Milwaukee, WI (US)

Correspondence Address: STOUT, UXA, BUYAN & MULLINS LLP, 4 VENTURE, SUITE 300, IRVINE, CA, 92618, US

(73) Assignee: The Regents of the University, Oakland, CA (US)

(21) Appl. No.: 12/066,381

(22) PCT Filed: Sep. 12, 2006
(86) PCT No.: PCT/US06/35789

§ 371 Date: Nov. 21, 2008

Related U.S. Application Data
(60) Provisional application No. 60/716,859, filed on Sep. 13, 2005.

(51) Int. Cl. A61K 31/415; A61K 31/41; A61P 9/10

(52) U.S. Cl. 514/381; 514/406

(57) ABSTRACT

Methods for treating or preventing atherosclerosis in human or non-human animal subjects by inhibiting or blocking intermediate-conductance calcium activated potassium channels associated with vascular smooth muscle and/or other cells which play a role in the pathogenesis of atherosclerosis (e.g., KCa3.1, KCNN4, IKCa1, IK1, SK4 channels).

### RELATED APPLICATION

This patent application claims priority to U.S. Provisional Patent Application Ser. No. 60/716,859 filed Sep. 13, 2005, the entirety of which is expressly incorporated herein by reference.

### STATEMENT REGARDING FEDERALLY SPONSORED RESEARCH

This invention was made with Government support under Grants HL65203 and HL62852 awarded by the National Institutes of Health as well as Veterans Administration Merit Award Grant Program 36 by the Department of Veterans Affairs. The Government may have certain rights in this invention.

### FIELD OF THE INVENTION

The present invention relates generally to the fields of biology and medicine and more particularly to compositions and methods for treating or preventing atherosclerosis.

### BACKGROUND

A group of drugs knows as “statins” have become widely used as cholesterol-lowering agents. Statins act by competitively inhibiting HMG-CoA reductase, an enzyme of the metabolic pathway by which the body synthesizes cholesterol. Commercially available statin drugs include atorvastatin (Lipitor®), fluvastatin (Lescol®), lovastatin (Mevacor®, Altocor®), pravastatin (Pravacol®, Selektine®, Lipostat®), rosuvastatin (Crestor®) and simvastatin (Zocor®, Lipex®).

It has been suggested that statins are the most promising drugs to prevent the development or progression of atherosclerosis due to their cholesterol lowering effect in combination with other beneficial effects including stabilization of plaques, vascular protective effects, anti-proliferative and migratory effects, anti-inflammatory effects, and anti-oxidative effects. However, multiple clinical studies revealed that the reduction in cardiac events in subjects with coronary risk factors by statins is only 30%. In addition, statins have been associated with side effects such as muscle symptoms or myopathies (e.g., Myalgia—muscle ache or weakness without elevation of creatine kinase (CK) and/or Myositis—muscle ache or weakness with increased CK levels and Rhabdomyolysis—muscle symptoms with marked elevation of CK as well as creatinine elevation and hepatotoxicity). There are also certain contraindications to the use of at least some statin drugs, such as cholestasis, active liver disease or the concomitant administration of certain drugs that increase the potential for serious myopathy.

Thus, there remains a need for the development of new potent drugs for the treatment or prevention of athersclerosis without the potential for the side effects associated with statin therapy (e.g., rhabdomyolysis or injury to cardiac muscles) and/or for use in subjects for whom statin drug therapy is contraindicated.

A change of expression in calcium-activated potassium channels (KCa) from large conductance KCa (BKCa=KCa1.1) to intermediate conductance KCa (IKCa1=KCa3.1) occurs concomitantly with the phenotypic change of VSMCs from contractile to proliferative; a key process of vascular remodeling during atherosclerosis. Therefore, Applicants have hypothesized that up-regulation of IKCa1 activity plays a critical role in the progression of atherosclerosis. Compounds that may effectively inhibit IKCa1 activity have previously been described in U.S. Pat. No. 6,903,375 (Chandy et al.) entitled Non-Peptide Inhibition Of T-Lymphocyte Activation And Therapies Related Thereto, which is expressly incorporated herein by reference.

Included among the compounds known to effectively inhibit activity of IKCa1 is 1-[(2-chlorophenyl)diphenylmethyl]-1H-pyrazole (TRAM-34). TRAM-34 inhibits KCa3.1 channels which are predominantly expressed in proliferative VSMCs, activated T cells and macrophages but not in contractile VSMCs and non-activated inflammatory cells, leading to the selective anti-proliferatory and anti-inflammatory effects, and consequent vascular protective effect. In addition, appropriate levels of plasma cholesterol are still controversial, although short-term treatment with statins has been reported to reduce the incidence of ischemic cardiac events in subjects with normal cholesterol levels by about 30%, KCa3.1 inhibiting compounds such as TRAM-34 may offer advantages over statin drugs or other therapies in preventing or treating atherosclerosis in non-hyperlipidemic patients.

### SUMMARY OF THE INVENTION

The present invention provides methods for treating or preventing atherosclerosis in human or animal subjects. These methods generally comprise the step of inhibiting or blocking intermediate-conductance calcium activated potassium channels (e.g., KCa3.1, KCNN4, IKCa1, IK1, SK4) located in vascular smooth muscle cells or other tissues associated with the pathogenesis of atherosclerotic lesions. Such inhibition or blocking of intermediate-conductance calcium activated potassium channels may be accomplished by administering to the subject an effective amount of a substance that comprises a compound that inhibits or blocks intermediate-conductance calcium activated potassium channels. Compounds that may be effective for this purpose include those having the structural formula:

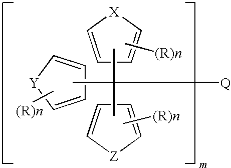

wherein,
X, Y and Z are same or different and are independently selected from CH2, O, S, NR₁, N═CH, CH═N and R₂—C═C—R₃, where R₂ and R₃ are H or may combine to form a saturated or unsaturated carbocyclic or heterocyclic ring, optionally substituted with one or more R groups;
R₁ is selected from H, alkyl, alkenyl, alkynyl, cycloalkyl, aryl, acyl and aroyl, optionally substituted with hydroxy, amino, substituted amino, cyano, alkoxy, halogen, trihaloalkyl, nitro, thio, alkylthio, carboxy and alkoxycarbonyl groups;
R is selected from H, halogen, trihaloalkyl, hydroxy, acyloxy, alkoxy, alkenyloxy, thio, alkylthio, nitro, cyano, ureido, acyl, carboxy, alkoxycarbonyl, N—(R₄)(R₅) and saturated or unsaturated, chiral or achiral, cyclic or acyclic, straight or branched hydrocarbyl group with from 1 to 20 carbon atoms, optionally substituted with hydroxy, halogen, trihaloalkyl, alkylthio, alkoxy, carboxy, alkoxycarbonyl, oxoalkyl, cyano and N—(R₄)(R₅) group,
R₄ and R₅ are selected from H, alkyl, alkenyl, alkynyl, cycloalkyl and acyl or R₄ and R₅ may combine to form a ring, wherein a carbon may be optionally substituted by a heteroatom selected from O, S or N—R₆,
R₆ is H, alkyl, alkenyl, alkynyl, cycloalkyl, hydroxyalkyl or carboxyalkyl,
n is 1-5; m is 1 or 2; with the proviso that
when m is 1, Q is selected from OH, CN, carboxyalkyl; N—(R₇)(R₅), where R₇ and R₈ are selected from H, lower alkyl (1-4C), cycloalkyl, aryl, acyl, amido, or R₇ and R₆ may combine to form a saturated or unsaturated heterocylic ring and optionally substituted with up to 3 additional heteroatoms selected from N, O, and S; or —NH-heterocycle, where the heterocycle is represented by thiazole, oxazole, isoxazole, pyridine, pyrimidine, and purine and
where U and V are selected from H and O; and

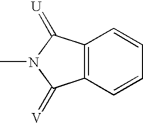

when m is 2, Q is a spacer of from 2-10 carbons as a straight or branched, chiral or achiral, cyclic or acyclic, saturated or unsaturated, hydrocarbon group, such as phenyl.

Further information regarding these compounds, and method for synthesis are described in U.S. Pat. No. 6,803,375 entitled Non-Peptide Inhibition Of T-Lymphocyte Activation And Therapies Related Thereto and copending U.S. patent application Ser. No. 10/533,060 entitled Compounds, Methods and Devices for Inhibiting Neoproliferative Changes in Blood Vessel Walls, both of which are expressly incorporated herein by reference.

In accordance with the present invention, non-limiting examples of compounds having the above-set-forth structural formula include but are not necessarily limited to: 1-[(2-chlorophenyl)diphenylmethyl]-1H-pyrazole (TRAM 34); 1-[(24-fluorphenyl)diphenylmethyl]-1H-pyrazole; 1-[(4-chlorophenyl)diphenylmethyl]-1H-pyrazole; 1-[(2-fluorphenyl)diphenylmethyl]-1H-pyrazole and 1-[(2-chlorophenyl)diphenylmethyl]-H-1,2,3,4-tetrazole.

Further in accordance with the invention, there are provided methods of the foregoing character wherein the substance administered to the subject substantially blocks or inhibits KCa3.1 channels that are predominantly expressed in proliferating vascular smooth muscle cells (VSMCs), endothelial cells, activated T cells and macrophages but not in contractile VSMCs. This selective KCa3.1 channel inhibition or blockade has a selective anti-proliferative and anti-inflammatory effect, and a consequent vascular protective effect.

Still further in accordance with the invention, substances that inhibit or block intermediate-conductance calcium activated potassium channels may be administered to the subject by any suitable route of administration including but not limited to injection or infusion (e.g., intravenous, intramuscular, subcutaneous), transdermal, transmucosal, via an implantable drug delivery device, etc.

### BRIEF DESCRIPTION OF THE DRAWINGS

The following detailed description and examples, and the accompanying figures, are intended to describe certain embodiments or examples of the invention and are not intended to limit the scope of the invention in any way.

FIGS. 1A-1C show differential expression of calcium-activated potassium channels in the human coronary microcirculation. FIG. 1A shows that IKCa1 protein expression is remarkably increased in subjects with coronary artery disease (CAD), compared to those without CAD. In contrast, BKCa expression is decreased in CAD subjects. Three subjects were examined in each group. The membrane protein samples (BKCa; 20 μg and IKCa; 40 μg) were analyzed by Western blot method (dilutions of primary antibodies; BKCa 1:500 and IKCa 1:1,000). FIG. 1B shows localization of IKCa1 protein using immunohistochemistry. a) In the tissue from non-CAD subjects (representative image from 47 year-old female with valvular disease), endothelial cells (ECs) were strongly stained, while the staining in VSMCs was faint. b) In the tissue from CAD subjects (52 year-old female with CAD), VSMCs showed strong immunostaining for IKCa1. c) There was no staining in the negative control. d) In an isolated small coronary artery (internal diameter ≈300 μm) from CAD subjects (71 year-old male with CAD), it is notable that VSMCs were heterogeneously stained. Positive staining appears in brown. Magnification 60×, (Antibody dilution; a and b 1:250 and d 1:160). L indicates lumen. Morphological changes in the human coronary microcirculation were examined by electron microscopy (FIG. 1C). Left panel) In vessels from non-CAD subjects, VSMCs are spindle shaped (arrowhead). Right panel) In vessels from CAD subjects, in the luminal overpopulations of VSMCs that appear in the tunica media, the cells are irregular in size and cubic in shape like cobblestones (blue arrow), whereas the main VSMCs are spindle shaped (red arrowhead). Magnification; 2,500×. Scale bars; 1 μm. L; lumen, E; endothelial cell, I; intimal layer, and M; medial layer.

FIGS. 2A and 2B show the induction of IKCa1 message by platelet-derived growth factor-BB (PDGF) in cultured human coronary artery smooth muscle cells (HCSMCs). FIG. 2A shows that IKCa1 mRNA expression is increased in response to PDGF treatment. FIG. 2B shows that Western blot analysis also revealed increased IKCa1 protein expression in HCSMCs after 48-hour stimulation with PDGF (40 μg membrane proteins, IKCa1 antibody 1:1,000 dilution).

FIGS. 3A-D show the inhibitory effects of TRAM-34 on proliferation and migration of cultured HCSMCs. FIG. 1A shows that TRAM-34 reduces the increase in cell number of HCSMCs in the presence of PDGF. FIG. 1B shows that the BrdU incorporation method revealed that PDGF-induced increase in DNA synthesis is also decreased by TRAM-34. FIG. 1C shows that treatment with TRAM-34 significantly inhibits c-fos up-regulation induced by PDGF (20 μg whole cell lysates and IKCa antibody 1:1,000 dilution). PDGF-induced VSMC migration is also inhibited by TRAM-34 (FIG. 1D).

FIGS. 4A-4C show IKCa1 up-regulation and VSMC migration in atherosclerotic lesions of apolipoprotein E (ApoE) knockout mice. FIG. 4A shows Western blot analysis indicating that IKCa1 channels are strongly expressed in aortas from ApoE knockout mice, whereas BKCa channels are down-regulated (IKCa; 40 μg membrane protein and 1:1,000 antibody dilution, and BKCa; 30 μg and 1:500). FIG. 4B shows that IKCa1 protein expression is restricted to the endothelial layer of aortas of wild type (WT) mice (panels a and c of FIG. 4B). In contrast, IKCa1 expression is extensively observed in aortic atherosclerotic lesions including ECs and migrated cells into the thickened intimal lesions (panel b of FIG. 4B). Note that VSMCs in luminal area of medial layer are also strongly stained (panel d of FIG. 4B). (antibody 1:100 dilution). FIG. 4C shows that the expression of SM α-actin is seen only in medial layer of aortas from wild type mice (panels a and c of FIG. 4C). In aortas of ApoE knockout mice, not only medial layer but also thickened intimal lesions are positively stained for SM α-actin (panel b of FIG. 4C). The stained areas in the intima overlap with those for IKCa1, indicating migrated VSMCs into the intima (panel d of FIG. 4C). (antibody 1:100 dilution).

FIGS. 5A and 5B show altered vasodilator response to KCa stimulation in ApoE KO mice. FIG. 5A shows an enhanced vasodilation to IKCa1 stimulation with EBIO in carotid artery segments of ApoE knockout mice. FIG. 54B shows that, in contrast, vasodilator response to BKCa stimulation with pimaric acid is reduced. # p<0.05 compared to wild type mice.

FIGS. 6A and 6B show the effects of long-term inhibition of IKCa1 activity on the progression of atherosclerosis in ApoE KO mice. FIG. 6A shows representative images of aortic atherosclerotic formation. In wild type mice, no formation of atherosclerotic lesions was observed. On the other hand, ApoE KO mice treated with vehicle displayed extensive atherosclerotic lesions throughout aortic trees from the aortic root to the iliac arteries, while a much smaller area was stained in the aorta from ApoE mice treated with TRAM-34. FIG. 6B shows that in summary, treatment with TRAM-34 markedly reduced the lesion area (atherosclerotic lesion area/whole aortic area) by approximately 60%.

FIG. 7 is a table (also referred to below as Table 1) showing the effects of long-term IKCa1 blockade by TRAM-34 on body weight, heart weight, systemic blood pressure, heart rate, and plasma cholesterol levels in mice.

### DETAILED DESCRIPTION AND EXAMPLES

The following detailed description and the accompanying drawings are intended to describe some, but not necessarily all, examples or embodiments of the invention. The contents of this detailed description do not limit the scope of the Invention in any way.

Unlike drugs that act by inhibiting cholesterol biosynthesis (e.g., statins) the treatments of the present invention act to prevent the development of atherosclerosis irrespective of the subjects plasma cholesterol levels. While some antihyperlipidemic agents (e.g., certain statins) have been reported to reduce the incidence of ischemic cardiac events even by approximately 30% in subjects with normal cholesterol levels, the treatments of the present invention (e.g., inhibiting or blocking intermediate-conductance calcium activated potassium channels (e.g., KCa3.1, KCNN4, IKCa1, IK1, SK4) may provide better means for treating subjects who exhibit symptoms of atherosclerosis, or are at risk for developing atherosclerosis, even though they may have normal or low plasma cholesterol levels.

Applicants have found that expression of the intermediate-conductance calcium activated potassium channel KCa3.1 (KCNN4, IKCa1, IK1, SK4) is significantly increased in T lymphocytes, macrophages and vascular smooth muscle cells from atherosclerotic lesions in both humans and mice with atherosclerosis. In cultured human coronary artery smooth muscle cells (HCSMCs) the platelet-derived growth-factor-BB (PDGF) increased proliferation and migration concomitant with an up-regulation of KCa3.1 (IKCa1). In view of this finding, Applicants tested whether KCa3.1 blockers, such as TRAM-34, could suppress the proliferation and migration of these cells thereby deterring the formation of atherosclerotic lesions.

Through the in-vitro studies described here below, Applicants have determined that TRAM-34, a KCa3.1 blocker, inhibited PDGF induced proliferation and migration of cultured HCSMCs. Additionally, Applicants tested whether TRAM-34 would prevent atherosclerosis development in the ApoE-knockout mouse, a widely used animal model of atherosclerosis. Long-term treatment with TRAM-34 reduced the development of atherosclerotic lesions (consisting of proliferating and migrating VSMCs, macrophages and T lymphocytes) in these mice by 60% compared to ApoE KO mice treated with vehicle (peanut oil) when the animals were fed a high-cholesterol diet. An nitric oxide-mediated component of endothelium-dependent vasodilation was restored in these animals due to the reduced superoxide production from VSMCs. Plasma levels of macrophage chemoattractants (MCP-1 and TNF-alpha) were also reduced, concomitant with the decreased accumulation of macrophages in the plaques. These results demonstrate that KCa3.1 blockade constitutes a novel therapeutic approach to the prevention and treatment of atherosclerosis.

### Materials and Methods

Tissue acquisition: Human coronary arteries. Human small coronary arteries (n=26) were isolated as reported previously. Procedures for harvesting tissue samples were in accordance with guidelines established by the local Institutional Review Boards. Mouse caromid vessels. Mice anesthetized with sodium pentobarbital (50 mg/kg, i.p. Abbott Laboratories, North Chicago, Ill.) were sacrificed by collecting blood from the hearts. Under a microscope, 1st˜2nd branches of external carotid arteries (150˜250 μm in internal diameter, 1-2 mm in length) were carefully removed and placed immediately into cold (4° C.) HEPES buffer.

Western blot analysis: Total cell lysates or membrane fractions were harvested and protein samples separated on an electrophoresis gel by SDS-PAGE and then transferred to a PVDF membrane. The gels were stained in Coomassie blue to confirm equal protein loading. Membranes were blocked with 10% nonfat dried milk, blotted with primary antibodies (BKCa α-subunit [Affinity BioReagents], c-fos [Santa Cruz, Inc.] and IKCa) and subsequently probed with a horseradish peroxidase-labeled donkey anti-rabbit antibody (1:5,000˜10,000 [Santa Cruz, Inc.]). The bound antibody was detected by chemiluminescence (ECL Plus, Amersham). The polyclonal primary antibody against human and mouse IKCa was obtained from sera of rabbits immunized using oligopeptides with following amino acids sequences; H-LNASYRSIGALNQVRC-NH2 (S4-5 of human and mouse IKCa).

Immunohistochemistry: Immunohistochemistry was performed to localize IKCa and SM α-actin in the blood vessels as previously described. Briefly, tissues were fixed, and frozen in OCT compound. Sections (8 μm thick) were immunolabelled with primary antibodies (IKCa and SM α-actin [AnaSpec, Inc.]). Immunostains were visualized by Vectastain Universal Quick kit, Vector Laboratories. As a control for non-specific binding, the primary antibody was omitted.

Electron microscopy: Electron microscopy was performed as previously reported.

Cell culture: Human coronary artery smooth muscle cells (HCSMCs, Camblex, inc.) were maintained according to manufacturer's instructions. To achieve a quiescent state, cells were incubated in serum-free SmBM for 48 hours. All experiments were performed between passages 5 and 7.

Real-time PCR: HCSMCs were seeded onto 6-well plates at a density of 12×10⁴/well in SmGM-2 and cultured up to 70% confluence (3 days). After achieving a quiescent state, cells were stimulated for 48 hours with or without 20 ng/ml platelet-derived growth factor-BB (PDGF, R&D Systems, Minneapolis, Minn.). RNA was isolated with TRIZOL Reagent (Invitrogen), reverse-transcribed to cDNA with iScript cDNA synthesis kit (Bio-Rad). Real-time PCR (icycler, Bio-Rad) was used for quantification of transcripts for hIKCa (Gen bank Accession No. NM 002250) and GAPDH (AF 100860) using iQ SYBR Green Supermix (Bio-Rad). Primers were designed (Beacon Designer software 3.0, PREMIER Biosoft International, Palo Alto, Calif.) and synthesized (Integrated DNA Technologies, Inc., Coralville, Iowa) as follows: for hIKCa, 5′-GGC CAA GCT TTA CAT GAA CAC G-3′ (sense) and 5′-GTC TGA AAG GTG CCC AGT GG-3′ (antisense); for GAPDH, 5′-CCT GCC AAG TAT GAT GAC-3′ (sense) and 5′-GGA GTT GCT GTT GAA GTC-3′ (antisense). Each 25 □l PCR reaction consisted of 10⁻⁷ M forward and reverse primers. The reaction conditions were as follows: 3 minutes at 95° followed by 40 cycles at 95° for 60 seconds, 60° for 60 seconds. All reactions were carried out in duplicate and included no template controls. Threshold cycles (Ct) were calculated by iCycler iQ (Bio-Rad). Real-time RT-PCR signals for hIKCa were standardized to GAPDH by use of the equation CtX−Ct_{rGAPDH}=ΔCt. Relative quantification and the fold change were calculated according to the formula ΔCt_{w/o}−ΔCtX=ΔΔCt and 2^{ΔΔct} respectively (w/o=without stimulus).

Cell proliferation assays: Cell proliferation assays were performed as previously reported. Briefly, quiescent HCSMCs seeded at a density of 4×10⁴/well in 6-well plates were stimulated by 20 ng/mL PDGF in the presence or absence of 10⁻⁷ M TRAM-34, a selective IKCa blocker. Forty eight hours after stimulation, the number of cells was counted with a hemocytometer (MARIENFELD, Lauda-Konigshofen Germany). In another set of experiments, a BrdU cell proliferation assay was also performed with quiescent cells in 96-well plates at a density of 1×10⁴/well according to the manufacturer's instructions (Colorimetric Cell Proliferation ELISA, Roche, Penzberg Germany). In this study, BrdU (10⁻⁵ M in medium) was applied 24 hours prior to the measurements.

Cell migration assay: A Cell migration assay was carried out with the Transwell system (Corning, Acton, Mass.) as previously reported. Briefly, cells (3×10⁵ cells/mL) were seeded onto the upper chamber of Transwells, and the lower chamber was filled with serum-free medium containing 20 ng/ml PDGF. TRAM-34 (10⁻⁸˜10⁻⁷ M) was added to both chambers. After 8-hour stimulation, migrated cells were fixed and stained with the Diff-Quick Stain (IMEB Inc. Chicago, Ill.) and counted under a microscope.

Mouse treatment: C57BL/6J male mice (wild type [WT] n=11 and ApoE deficient type [EKO] n=38, The Jackson Laboratory) were used. EKO mice were weaned at 4 weeks of age onto a high-cholesterol diet (1.3% cholesterol; TD 96121, Harian/Teklad) and treated with daily subcutaneous injection of TRAM-34 (120 mg/kg/day) or vehicle (peanut oil) for 12 weeks. Littermate war mice were used as the control group in the experiments. Mice were provided diet and water ad libitum and maintained on a 12-hour light/dark cycle. All animal experiments were conducted according to the Guidelines for Animal Experiments at Medical College of Wisconsin.

Hemodynamic analysis of mice: At 16 weeks of age, mice were anesthetized, and right femoral arteries were cannulated for continuous measurement of arterial pressure and heart rate (pressure transducer; Bioresearch Center, Nagoya, Japan) and recorded continuously by computer for 30 min.

Plasma lipid analysis: Plasma was obtained by centrifugation of blood and stored at −80° C. until each assay was performed. Plasma cholesterol levels were analyzed by General Medical Laboratories (Madison, Wis.).

Histological analysis of atherosclerosis in mouse aortas: Isolation of aortas and quantification of atherosclerosis were performed as previously described. Briefly, aortas (from aortic arch to iliac bifurcation) were opened longitudinally, pinned onto a silicon-coated dish, fixed with 4% paraformaldehyde, and stained in 1.0% (v/w) Sudan III solution (The Science Company, Denver, Colo.). Images were acquired using a digital camera (C-755, Minolta), and the surface area of atherosclerotic lesions was measured as the percentage of total area of the opened aorta using imaging software, MetaMorph (Universal Imaging Corp).

Videomicroscopy: The preparation for videomicroscopy has been previously described. Vasomotor and endothelial function was confirmed by measuring constriction to 50 mM KCl and dilation to acetylcholine (ACh, 10⁻⁴ M, mouse vessels pressurized at 40 mmHg) or to bradykinin (10⁻⁷ mol/L, human vessels at 60 mmHg). Vessels were preconstricted with U46619 (10⁻⁹˜10⁻⁸ M for mouse vessels) or ACh (10⁻⁸˜5×10⁻⁷ M for human vessels) to adjust tone to a level between 30% to 50% of passive diameter. Dose-dependent vasodilation to 1-ethyl-2-benzimidazolinone (EBIO, an IKCa opener, 10⁻⁵˜10⁻⁴ M) and to pimaric acid, a BKCa opener (10⁻⁶˜10⁻⁵ M) were measured in isolated and pressurized vessels from human or mouse. In some experiments, endothelial cells (ECs) were denuded.

Statistical Analysis: All data are expressed as mean±SE. Data acquired by either real-time PCR, cell proliferation and migration assays, or histological analysis of atherosclerotic lesion were compared by using paired Student's t test. Percent dilation was calculated as the percent change from the preconstricted diameter to the passive diameter in Ca²⁺-free Krebs containing 10⁻⁴ M papaverine. Percent constriction or basal tone was determined by calculating the percent reduction in the passive diameter. To compare dose-response relationships between treatment groups, a two-way ANOVA supported by a Bonferroni post hoc test was used. Statistical comparisons of maximal percent vasodilation and basal tone under different treatments were performed by paired Student's t test. All procedures were done using ‘proc mixed’ or ‘proc gim’ programs of SAS for Windows version 8.2. Statistical significance was defined as a value of P<0.05.

### Results

Differential Expression of KCa and Morphological Changes in Diseased Human Coronary Microvessels

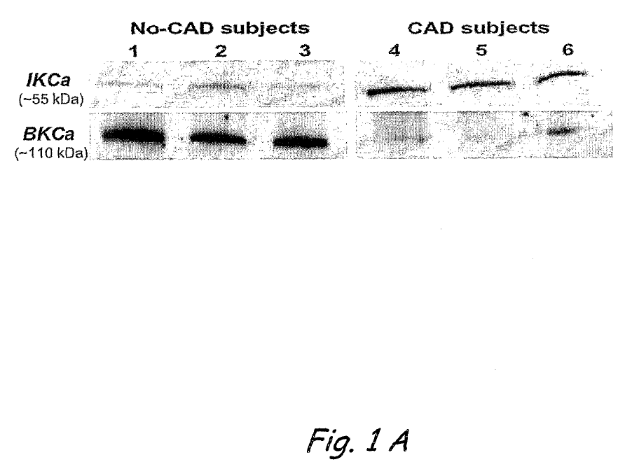

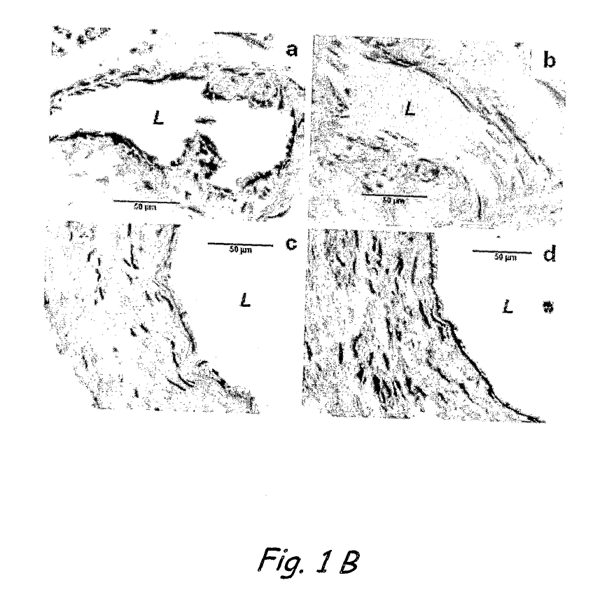

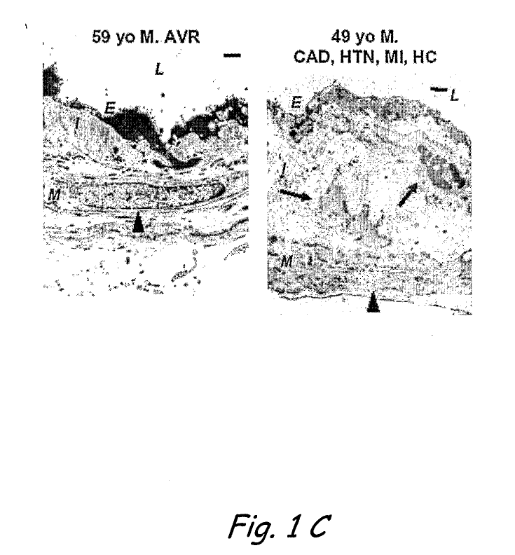

IKCa1 protein expression was markedly increased in small coronary arteries from subjects with coronary artery disease (CAD) compared to those from subjects without CAD. In contrast, BKCa expression was comparatively decreased in CAD subjects (FIG. 1A).

Immunohistochemistry demonstrated that endothelial cells (ECs) were positively stained for IKCa protein in vessels (≈100 μm in diameter) from subjects without CAD, while VSMCs showed little staining (FIG. 1B-a). In subjects with CAD, VSMCs showed marked staining (FIG. 1B-b). In a larger artery (internal diameter=−300 μm) from a subject with CAD, heterogeneous staining was observed among VSMCs of the medial layer (FIG. 1B-d).

Morphological changes in vessels were examined by electron microscopy. Microvessels from subjects without CAD displayed a single endothelial layer and two layers of spindle-shaped VSMCs (arrowhead) with extracellular spaces narrow and regular in width, representing normal architecture (FIG. 1C left panel). In vessels from subjects with CAD (FIG. 1C right panel), the medial layer was thickened and included spindle-shaped VSMCs and irregularly-shaped and disarranged VSMCs surrounded by excess extracellular matrix. Elastic components between ECs and VSMCs became thicker and continued on to the inner elastic lamina. These findings provide morphological evidence of VSMC phenotypes present in the human coronary microcirculation in atherosclerosis. Taken together, these results support the hypothesis that IKCa1 up-regulation is involved in the morphological or phenotypic changes of VSMCs in atherosclerosis in humans.

Role of IKCa1 in VSMC Proliferation and Migration In Vitro

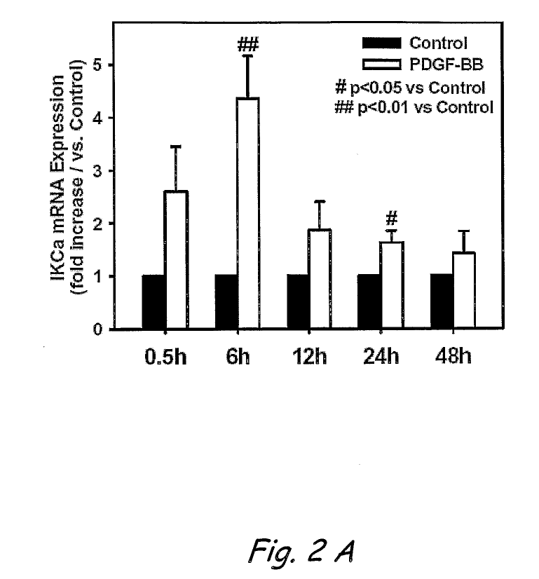

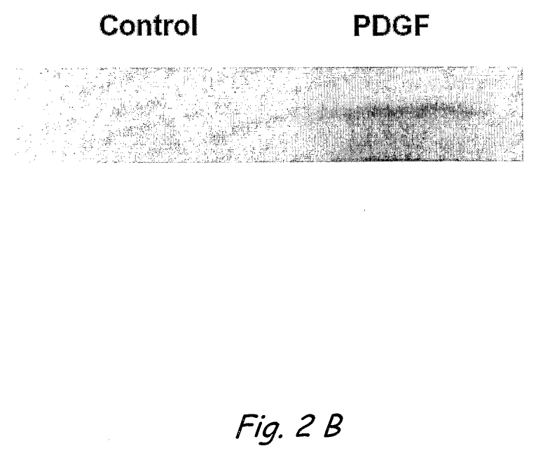

IKCa1 expression was determined during VSMC proliferation in response to PDGF in cultured HCSMCs. Real-time RT-PCR showed that PDGF increased IKCa mRNA expression in a time-dependent manner (Max response at 6 h, 4.2±1.0-fold, p<0.05 vs Control, n=5) (FIG. 2A). Western blot analysis also revealed that membranous expression of IKCa proteins was increased after 48-hour exposure to PDGF (FIG. 2B). BKCa expression was not detectable before or after treatment with PDGF. These findings suggest that IKCa1 up-regulation is concomitant with the progression of VSMC proliferation.

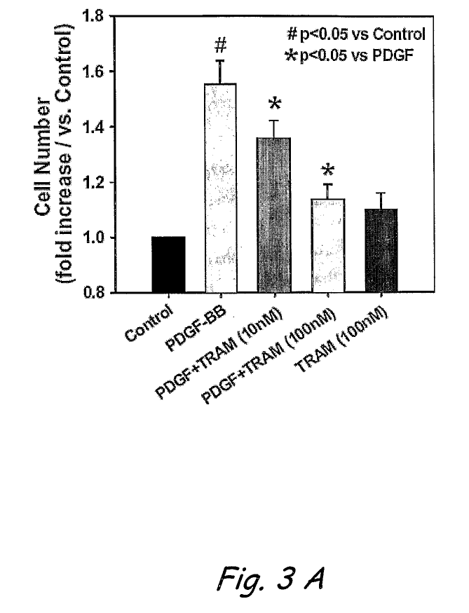

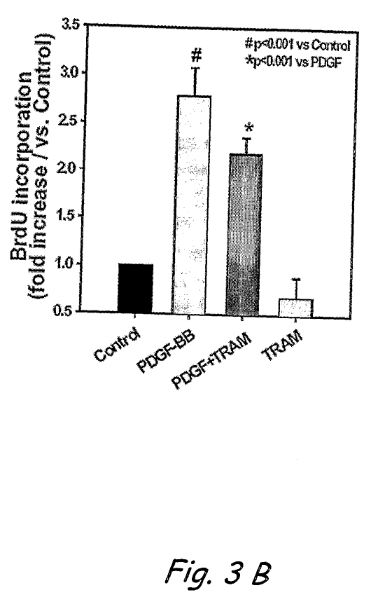

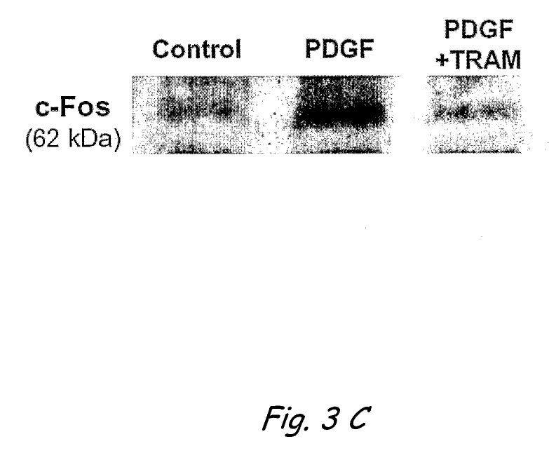

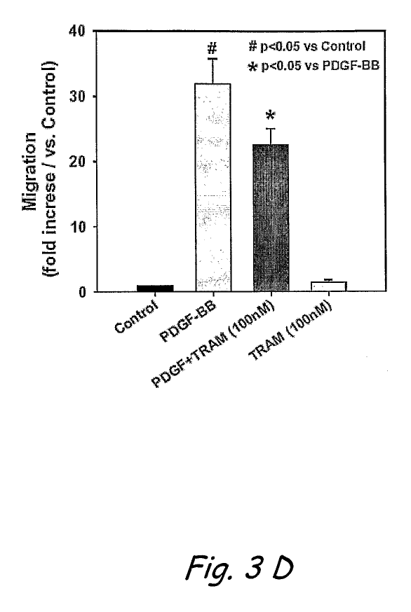

The role of IKCa1 in cultured HCSMC proliferation was examined by blocking the channel activity with TRAM-34, a selective IKCa1 blocker. FIG. 3A shows the effect of blocking IKCa activity with TRAM-34 on PDGF-stimulated HCSMC proliferation. Treatment of HCSMC for 48 hours in the presence of PDGF induced a significant increase in cell number (PDGF alone; 1.6±0.1-fold of control, n=7). The proliferation was significantly reduced by TRAM-34 in a dose-dependent manner (PDGF+TRAM-34; 1.1±0.1-fold of control at 10⁻⁷ M, p<0.05 vs PDGF alone, n=7). TRAM-34 in the absence of PDGF had no effect on HCSMC proliferation, Glibenclamide, an ATP-sensitive potassium channel blocker had no effect on PDGF-induced HCSMC proliferation (data not shown, n=4). Treatment with either PDGF alone, PDGF+TRAM-34, or TRAM-34 alone did not affect cell viability. The role of IKCa activity in DNA synthesis was determined by BrdU incorporation assay (FIG. 3B). PDGF significantly increased DNA synthesis in HCSMCs (PDGF alone; 2.8±0.3-fold of control, n=26). TRAM-34 suppressed PDGF-BB-induced DNA synthesis of HCSMCs (PDGF+TRAM-34; 2.2±0.2-fold of control, p<0.05 vs PDGF alone, n=26). TRAM alone had no effect on DNA synthesis (n=6).

To provide additional support for the inhibitory effect of IKCa1 blockade on cell proliferation and DNA synthesis, the expression of c-fos, a proto-oncogene intimately involved in cell proliferation, was examined in HCSMCs. PDGF induced up-regulation of c-fos protein in HCSMCs (FIG. 3C) that was markedly reduced by TRAM-34.

A transwell migration assay was employed to test the role of IKCa in VSMC migration. As shown in FIG. 3D, PDGF stimulated HCSMC migration (32±4-fold of control n=10). TRAM-34 inhibited PDGF-induced migration (PDGF+TRAM-34; 2392-fold of control n=4, p<0.05 vs PDGF alone). These findings indicate that increases in IKCa1 expression and activity are associated with VSMC proliferation and migration, a key step in the early stage of the development of atherosclerosis.

Up-Regulation of IKCa1 in Atherosclerotic Mouse Aortas

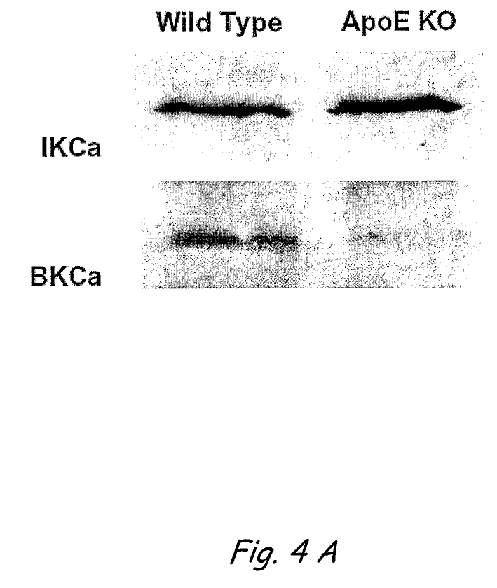

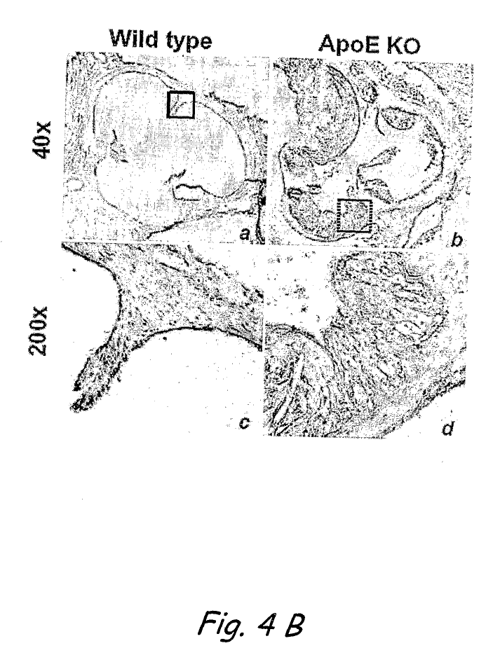

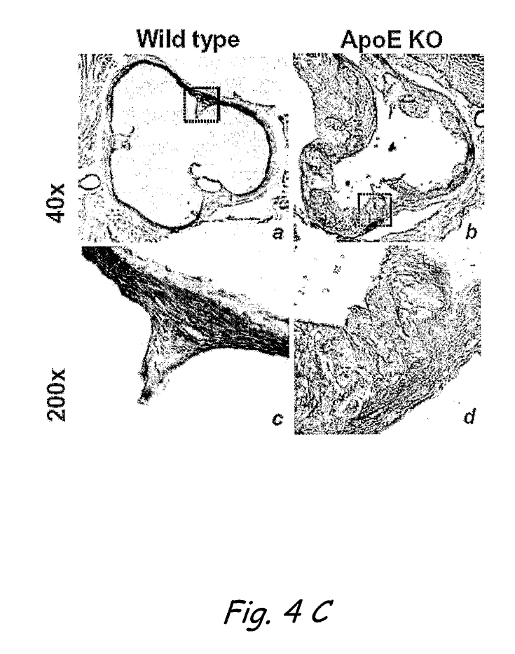

The expression of IKCa1 and BKCa were examined in ApoE KO mice. IKCa protein was increased and BKCa reduced in aortas of ApoE KO mice (FIG. 4A). Endothelial denudation did not alter the differential expression of KCa in mouse aortas (data not shown).

The localization of IKCa1 was examined by immunohistochemistry. As shown in FIG. 4B, IKCa protein was localized in the endothelial layer in aortas of WT mice, whereas IKCa were detected in the endothelial layer, intimally-migrated cells, and some VSMCs in the luminal area of medial layer in aortas of ApoE KO mice.

SM α-actin localization was determined in mouse aortas (FIG. 4C). While only VSMCs in the medical layer were positively stained in aortas of WT mice (FIG. 4C-a and c), SM α-actin expression was observed both in the medial layer and in the intimal atherosclerotic lesions in those of ApoE-KO mice (FIG. 4C-b and d). The intimal staining overlapped with that for IKCa1 (FIGS. 4B-d and 4C-d), indicating the presence of intimally-migrated VSMCs, which express IKCa1. Thus, IKCa1 up-regulation in atherosclerotic vessels results from VSMCs that proliferate and migrate into the intima.

Differential Activity of KCa in Vessels from Atherosclerotic Subjects

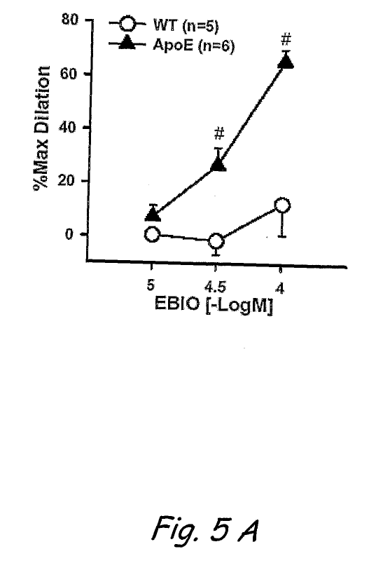

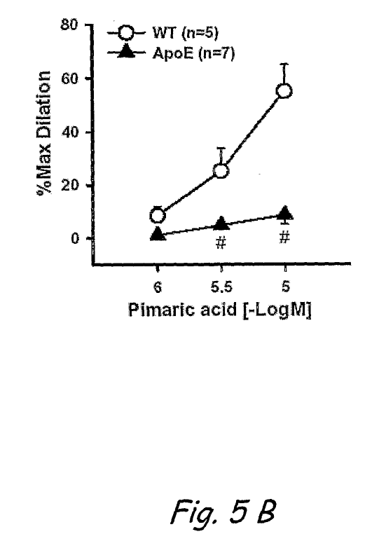

In endothelium-denuded mouse carotid artery segments, little dilation to EBIO, an IKCa1 opener was observed in WT mice (% max. dilation; 13±12% at 10⁻⁴ M), while the vasodilation was significantly enhanced in ApoE KO mice (66±4% p<0.05 vs WT) (FIG. 5A). In contrast, pimaric acid, a BKCa opener elicited potent vasodilation in WT mice in a dose-dependent manner (% max. dilation; 55±10% at 10⁻⁵ M), but the dilation was markedly reduced in ApoE KO mice (9±3% p<0.05 vs WT) (FIG. 5B).

When patients were stratified according to the presence or absence of CAD (no CAD [57±13y.o.] n=8 and CAD [65*11y.o.] n=12), vasodilation of human coronary arterioles to EBIO was identical between the groups (% max. dilation; no CAD 59±12 and CAD 61±8% at 10⁻⁴ M). However, endothelial denudation significantly reduced the dilation only in vessels from non-CAD subjects (no CAD 22±14 vs CAD 58±9%, p<0.05). Vasodilation of endothelium-denuded vessels to 3×10⁻⁶ M pimaric acid in CAD subjects (31±3%, p<0.05 vs non CAD, n=3) was significantly lower than that in non-CAD subjects (56±6%, n=3). These results suggest greater IKCa1 activity and relatively less BKCa activity in VSMCs of vessels in humans and mouse with atherosclerosis, consistent with the differential expression of KCa.

Role of IKCa1 in the Development of Atherosclerosis in ApoE Knockout mice In Vivo

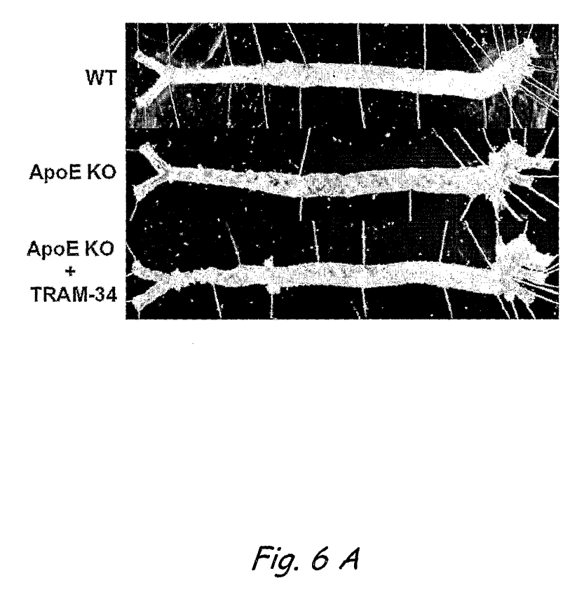

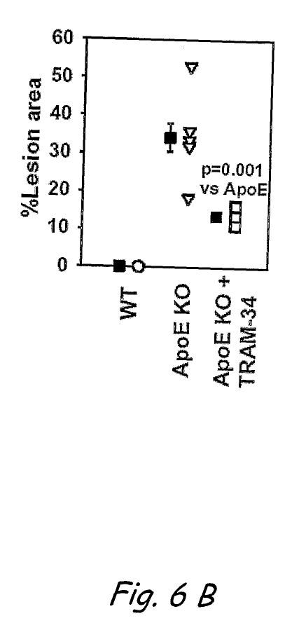

The effect of long-term IKCa1 blockade on the development of atherosclerosis was determined in mice. Representative images of aortic atherosclerotic lesions (stained in yellow˜orange) are shown in FIG. 6A. In ApoE KO mice treated with vehicle, atherosclerotic lesions were observed extensively from the aortic root to the iliac arteries. In ApoE KO mice treated with TRAM-34, much less staining was observed but in a similar distribution along the aorta. Quantitative measurements of atherosclerotic lesions are summarized in FIG. 6B. Aortas of ApoE KO mice displayed extensive lesions of atherosclerosis with 34±4% (18 to 53% n=6, p<0.05 vs WT) of lesion area (atherosclerotic lesion area/whole aortic area), while no lesions were seen in WT mice (0%, n=3). Treatment with TRAM-34 significantly reduced % lesion area approximately by 60% (14*1%, 11 to 17% n=7, p=0.001 vs ApoE KO mice treated with vehicle). Thus, IKCa1 activity plays an important role in the development of atherosclerosis.

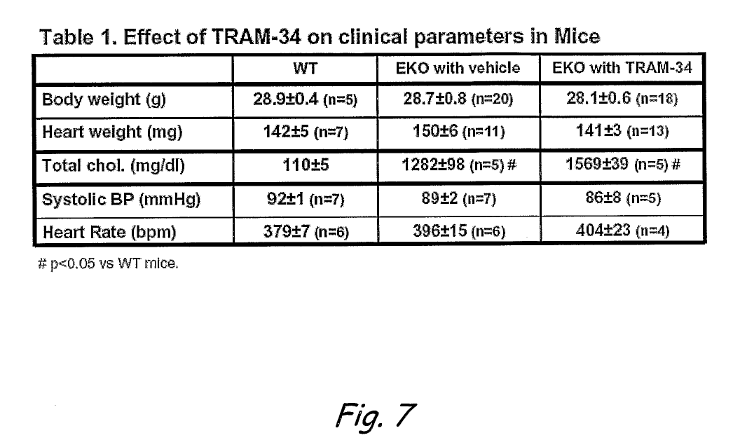

The effects of long-term IKCa1 blockade with TRAM-34 on body weight, heart weight, systemic blood pressure, heart rate, and plasma cholesterol levels are shown in FIG. 7 (Table 1). One mouse in each group (vehicle or TRAM-34) died due to unknown reasons during the 14-week treatment. Plasma cholesterol levels were higher in ApoE KO mice treated with vehicle or TRAM-34 than in WT mice, while there was no significant difference of cholesterol levels between ApoE KO mice treated with vehicle and those with TRAM-34. There were no significant differences of body and heart weight among the groups. Blood pressure and heart rate were also unaltered by the treatment.

Summary and Discussion

This study examines the role of IKCa1 in the development of atherosclerosis. The findings are four-fold. First, IKCa1 expression and activity are increased in the coronary circulation of patients with CAD and in aortas from mice with atherosclerosis. BKCa are down-regulated under the same conditions. Second, the increased expression of IKCa1 is associated with the proliferation and migration of VSMCs, macrophages and T lymphocytes in vivo and in vitro. Third, blockade of IKCa1 activity inhibits proliferation and migration of HCSMCs by suppressing c-fos expression and DNA synthesis. Finally, long-term IKCa1 blockade inhibits the development of atherosclerosis in mice. Taken together, these findings demonstrate that up-regulation of IKCa1 activity plays a crucial role in the proliferation and migration of VSMCs and inflammatory cells, an early step in the development of atherosclerosis and suggests that IKCa1 channels are a potential therapeutic target for preventing vascular morphological remodeling during atherosclerosis.

IKCa1 Up-Regulation in Proliferatory and Migratory VSMCs

Recent in-vivo studies demonstrated IKCa up-regulation during the process of vascular remodeling (VSMC proliferation) following myocardial infarction or chronic inhibition of NO synthesis in rats and rabbits. Other investigators also reported IKCa1 up-regulation in VSMCs migrated to neointima in carotid arteries following balloon catheter injury (Kohler et al). In the present study, we found that IKCa expression is increased in proliferating VSMCs in atherosclerotic vessels and in cultured HCSMCs stimulated with PDGF-BB. This is consistent with results reported by Neylon et al who demonstrated in cultured rat aortic SMCs that enhanced IKCa activity is closely related to cellular proliferative rate. In addition, IKCa are up-regulated and critically participate in the process of proliferation and migration in a variety of activated cells including activated T cells, macrophages and cancer cells. Thus, IKCa may serve a fundamental role in cellular activation common among several cell types.

Role of IKCa1 in Cellular Proliferation

In the present study, PDGF-induced HCSMC proliferation was inhibited with TRAM-34 in vitro. Similarly the proliferation of rat aortic VSMC cell lines induced by epidermal growth factor is blocked by IKCa1 blockers. IKCa1 blockers also inhibit the proliferation of cancer cells, T and B cells. The intracellular calcium concentration ([Ca²⁺]i) plays a critical role in initiating and maintaining the cellular activation process through the regulation of intracellular signaling cascades. Ca²⁺ influx through voltage-gated calcium channels and Ca²⁺ release from ryanodine receptors in response to mitogens initiates the activation of the mitogen-activated protein kinase (MAPK)/extracellular signal regulated kinase (ERK1/2) cascade followed by the activation of transcription factors, induction of early response genes and DNA synthesis concomitant with phenotypic changes in VSMCs. An increase in [Ca²⁺]i following membrane depolarization by high extracellular concentration of KCl induces VSMC differentiation marker genes via activation of Rho kinases. However, it is unlikely that membrane depolarization by blockade of IKCa1 with TRAM-34 inhibits the early process of VSMC proliferation, since very few IKCa1 channels are expressed in contractile or quiescent VSMCs.

Neylon et al reported evidence for differential membrane potentials from contractile and proliferative VSMC phenotypes. Contractile VSMCs, which express BKCa, have less negative resting membrane potential than proliferative VSMCs, which express IKCa1. In contractile VSMCs, exposure to endothelin-1 induces an elevation in [Ca²⁺]i and membrane depolarization, and pharmacological blockade of potassium channels does not modulate the depolarization. In contrast, when [Ca²⁺]i is elevated by the same agonists in proliferative VSMCs, there is a pronounced hyperpolarization due to the subsequent IKCa1 activation. IKCa1 plays a more important role than BKCa in shaping Ca²⁺ signals of proliferating cells, because of its higher Ca²⁺ affinity (EC₅₀ of IKCa1; ≈300 nM, BKCa; ≈6 μM). Indeed, IKCa1 up-regulation enhances the electrochemical driving force for Ca²⁺ influx through membrane hyperpolarization and thus sustains high [Ca²⁺] levels required for gene transcription to promote mitogenesis in lymphocytes, erythrocytes, and fibroblasts. These data suggest that IKCa1 channels actively participate in the regulation of cell proliferation by controlling [Ca²⁺]i and subsequently regulating the activities of Ca²⁺/calmodulin-dependent protein kinases and transcription factors responsible for mitogenesis. Thus, blockade of IKCa1 may reduce [Ca²⁺]i, leading to the inhibition of mitogenesis and VSMC proliferation, thereby producing an anti-atherosclerotic effect.

Alternative Mechanisms for the Anti-Atherosclerotic Effect of IKCa1 Blockade

It has been reported that proliferative VSMCs generate more reactive oxygen species (ROS) such as superoxide than contractile VSMCs, which might scavenge nitric oxide released from ECs. Similar observations were observed in vivo, where reduced endothelium-dependent vasorelaxation is due to excess oxidative stress generated in the media of atherosclerotic rabbit aortas. ApoE KO mice also exhibit reduced nitric oxide bioavailability. Thus, IKCa1 blockade might act by reducing oxidative stress and preserving nitric oxide bioavailability. However, IKCa1 channels also play an important role in the function of macrophages and T cells, and it is thus likely that inhibition of atherogenic inflammatory processes contributes to the anti-atherosclerotic effect of IKCa1 blockade.

It is to be appreciated that the invention has been described hereabove with reference to certain examples or embodiments of the invention but that various additions, deletions, alterations and modifications may be made to these examples and embodiments without departing from the intended spirit and scope of the invention. For example, any element or attribute of one embodiment or example may be incorporated into or used with another embodiment or example, unless otherwise indicated and/or unless doing so would render the embodiment or example unsuitable for its intended use. Also, where steps of a method or process have been described or recited in a certain order, the order of such steps may be changed unless otherwise indicated and/or unless doing so would render the method or process unsuitable for its intended use. All reasonable additions, deletions, modifications and alterations are to be considered equivalents of the described examples and embodiments and are to be included within the scope of the following claims.

What is claimed is:

1. A method for treating or preventing atherosclerosis in a human or non-human animal subject, said method comprising the step of inhibiting or blocking intermediate-conductance calcium activated potassium channels.

2. A method according to claim 1 wherein the step of inhibiting or blocking intermediate-conductance calcium activated potassium channels comprises inhibiting or blocking intermediate-conductance calcium activated potassium channels located in vascular smooth muscle cells or other tissues associated with the pathogenesis of atherosclerotic lesions.

3. A method according to claim 1 wherein the step of inhibiting or blocking intermediate-conductance calcium activated potassium channels comprises administering to the subject an effective amount of a substance that inhibits or blocks a calcium activated potassium channel selected from the group consisting of: KCa3.1, KCNN4, IKCa1, IK1 and SK4.

4. A method according to claim 3 wherein the substance comprises a compound having the structural formula: 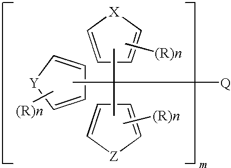
wherein,
X, Y and Z are same or different and are independently selected from CH2, O, S, NR₁, N═CH, CH═N and R₂—C═C—R₃, where R₂ and R₃ are H or may combine to form a saturated or unsaturated carbocyclic or heterocyclic ring, optionally substituted with one or more R groups;
R₁ is selected from H, alkyl, alkenyl, alkynyl, cycloalkyl, aryl, acyl and aroyl, optionally substituted with hydroxy, amino, substituted amino, cyano, alkoxy, halogen, trihaloalkyl, nitro, thio, alkylthio, carboxy and alkoxycarbonyl groups;
R is selected from H, halogen, trihaloalkyl, hydroxy, acyloxy, alkoxy, alkenyloxy, thio, alkylthio, nitro, cyano, ureido, acyl, carboxy, alkoxycarbonyl, N—(R₄)(R₅) and saturated or unsaturated, chiral or achiral, cyclic or acyclic, straight or branched hydrocarbyl group with from 1 to 20 carbon atoms, optionally substituted with hydroxy, halogen, trihaloalkyl, alkylthio, alkoxy, carboxy, alkoxycarbonyl, oxoalkyl, cyano and N—(R₄)(R₅) group,
R₄ and R₅ are selected from H, alkyl, alkenyl, alkynyl, cycloalkyl and acyl or R₄ and R₅ may combine to form a ring, wherein a carbon may be optionally substituted by a heteroatom selected from O, S or N—R₆,
R₅ is H, alkyl, alkenyl, alkynyl, cycloalkyl, hydroxyalkyl or carboxyalkyl,
n is 1-5;
m is 1 or 2;
with the proviso that;
when m is 1, Q is selected from OH, CN, carboxyalkyl, N—(R₇)(R₈), where R₇ and R₈ are selected from H, lower alkyl (1-4C), cycloalkyl, aryl, acyl, amido, or R₇ and R₈ may combine to form a saturated or unsaturated heterocylic ring and optionally substituted with up to 3 additional heteroatoms selected from N, O, and S; or —NH-heterocycle, where the heterocycle is represented by thiazole, oxazole, isoxazole, pyridine, pyrimidine, and purine and
where U and V are selected from H and O; and 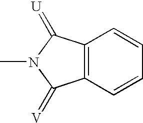
when m is 2, Q is a spacer of from 2-10 carbons as a straight or branched, chiral or achiral, cyclic or acyclic, saturated or unsaturated, hydrocarbon group, such as phenyl.

5. A method according to claim 3 wherein the compound is 1-[(2-chlorophenyl)diphenylmethyl]-1H-pyrazole.

6. A method according to claim 3 wherein the compound is 1-[(2-fluorphenyl)diphenylmethyl]-1H-pyrazole.

7. A method according to claim 3 wherein the compound is 1-[(4-chlorophenyl)diphenylmethyl]-1H-pyrazole.

8. A method according to claim 3 wherein the compound is 1-[(2-fluorphenyl)diphenylmethyl]-1H-pyrazole.

9. A method according to claim 3 wherein the compound is 1-[(2-chlorophenyl)diphenylmethyl]-1H-1,2,3,4-tetrazole.

10. A method according to any of claims 1-9 wherein the concentration of cholesterol in the subject's blood plasma is normal or subnormal.

11. A method according to any of claims 1-9 wherein the subject has previously been treated with a statin or other HMG-CoA Reductase inhibitor but has experienced side effects from such treatment.

12. A method according to claim 11 wherein the subject has previously been treated with a statin drug selected from the group consisting of. atorvastatin (Lipitor®), fluvastatin (Lescol®), lovastatin (Mevacor®, Altocor®), pravastatin (Pravacol®, Selektine®, Lipostat®), rosuvastatin (Crestor®) and simvastatin (Zocor®, Lipex®).

13. A method according to claim 11 wherein the subject has previously experienced symptoms of rhabdomyolysis or myopathy.

14. A method according to any of claims 1-9 wherein the subject has a contraindicating condition that contraindicates treatment with a statin or other 3-hydroxy-3-methylglutaryl coenzyme A reductase inhibitor.

15. A method according to claim 14 wherein the subject has a containdicating condition selected from the group consisting of: cholestasis, active liver disease and concomitant administration of drugs that increase the potential for serious myopathy.

16. The use of a composition that inhibits or blocks intermediate-conductance calcium activated potassium channels in the manufacture of a preparation for administration to humans or non-human animals for the treatment or prevention of atherosclerosis.

17. A use according to claim 16 wherein the composition comprises a compound according to any of claims 4-9.
